(19) United States
(12) Patent Application Publication
(10) Pub. No.: US 2015/0250641 A1
(43) Pub. Date: Sep. 10, 2015

(54) Anti-Snoring Apparatus and Method of Use

(71) Applicant: Lloyd Babineau, Lakeside, CA (US)

(72) Inventor: Lloyd Babineau, Lakeside, CA (US)

(21) Appl. No.: 14/201,786

(22) Filed: Mar. 7, 2014

(51) Int. Cl. A61F 5/56

(52) U.S. Cl. CPC A61F 5/56

(57) ABSTRACT

An anti-snoring apparatus with a clamshell body for attachment to a garment of a user. The anti-snoring apparatus promotes the user to sleep in a position other than on their back to prevent snoring. The clamshell body has first and second body portions that are secured together by a hinge clip The hinge clip has pivotal members which allow the user to open and close the clamshell. The hinge clip is pre-tensioned to bias the clamshell in a closed position. The pivotal members can be rotated so there are each positioned in a groove in one of the body portions, preventing them from contacting the user. Each body portion may include a pair of extensions which protrude outwardly from their respective body portion when secured thereon, and the protrusions may have different thicknesses.

### 1.0 TECHNICAL FIELD

The present invention relates to an anti-snoring apparatus that clips on the clothing of a user to prevent them from sleeping on their back. More, specifically the invention relates to an anti-snoring apparatus that can be quickly and securely clipped on the waistband of a user's clothing or undergarment to prevent them from sleeping on their back. It can be employed with any article of clothing having a waistband or a free upper end over which the apparatus can be clipped.

### 2.0 BACKGROUND

Anti-snoring devices of the prior art have previously employed removable members such as Eliam et al (U.S. Pat. No. 5,357,981). Eliam et al (hereinafter “Eliam”) discloses a removable anti-snoring device, which can be secured to a users undergarment. As seen in FIG. 3A of Eliam, there is a piece of fastening material fixedly attached to the garment of the user. The piece of hook-and-loop fastening material is a required to secure the anti-snoring device to the undergarment. This presents several distinct disadvantages. It would require the user employ a specific article of clothing to sleep since it must include the fastening material. Or it would require the user the tedious task of securing the fastening material to each and each and every garment that they would use for sleeping. The hook-and-loop fastener may also damage delicate clothing and collect lint and dust.

A need remains for an anti-snoring device that overcomes these disadvantages. The present invention addresses the deficiencies in the prior art.

### 3.0 SUMMARY

One aspect of the present invention is to provide an anti-snoring apparatus with a clamshell body and includes a hinge where the clamshell is opened by the user and then allowed to close about the waistband or free edge of the clothing of the user.

Another aspect of the present invention is to employ a hinge that is pre-tensioned and has enough tension to securely attach the apparatus to the clothing of the user. The hinge also allows for the body to close securely about waistbands or clothing with different thicknesses.

In yet another aspect of the present invention, the clamshell body may include removable members, which can be removed and replaced to adjust the height of the clamshell body.

In yet another aspect of the present invention, the hinge is provided by a hinge clip that secures the two portions of the clamshell body together. The hinge clip includes two pivotal members that to allow the user to open the clamshell body. These pivotal members can be moved into a position where they will not come into contact with the user when the apparatus is in use.

Other aspects of the invention are disclosed herein as discussed in the following Drawings and Detailed Description.

### 4.0 BRIEF DESCRIPTION OF THE DRAWINGS

The invention can be better understood with reference to the following figures. The components within the figures are not necessarily to scale, emphasis instead being placed on clearly illustrating example aspects of the invention. In the figures, like reference numerals designate corresponding parts throughout the different views and/or embodiments. It will be understood that certain components and details may not appear in the figures to assist in more clearly describing the invention.

FIG. 1 is a front perspective of an embodiment of the anti-snoring apparatus.

FIG. 2 is an exploded view of an embodiment of the anti-snoring apparatus.

FIG. 3 is a perspective view of a body portion of the anti-snoring apparatus.

FIG. 4 is a side-section view of an open position of the anti-snoring apparatus.

FIG. 5 is a side-section view of another position of the anti-snoring apparatus.

FIG. 6 is an enlarged elevation view of an embodiment of the anti-snoring apparatus in use.

FIG. 7 is an elevation view of an embodiment of the anti-snoring apparatus in use.

FIG. 8 is a front perspective of an embodiment of the anti-snoring apparatus.

FIG. 9 is a rear perspective of an embodiment of the anti-snoring apparatus.

FIG. 10 is a top view of an embodiment of the anti-snoring apparatus.

FIG. 11 is a bottom view of an embodiment of the anti-snoring apparatus.

FIG. 12 is a front view of an embodiment of the anti-snoring apparatus.

FIG. 13 is a rear view of an embodiment of the anti-snoring apparatus.

FIG. 14 is a left-side view of an embodiment of the anti-snoring apparatus.

FIG. 15 is a right-side view of an embodiment of the anti-snoring apparatus.

### 5.0 DETAILED DESCRIPTION OF EXAMPLE EMBODIMENTS

Following is a non-limiting written description of example embodiments illustrating various aspects of the invention. These examples are provided to enable a person of ordinary skill in the art to practice the full scope of the invention without having to engage in an undue amount of experimentation. As will be apparent to persons skilled in the art, further modifications and adaptations can be made without departing from the spirit and scope of the invention, which is limited only by the claims.

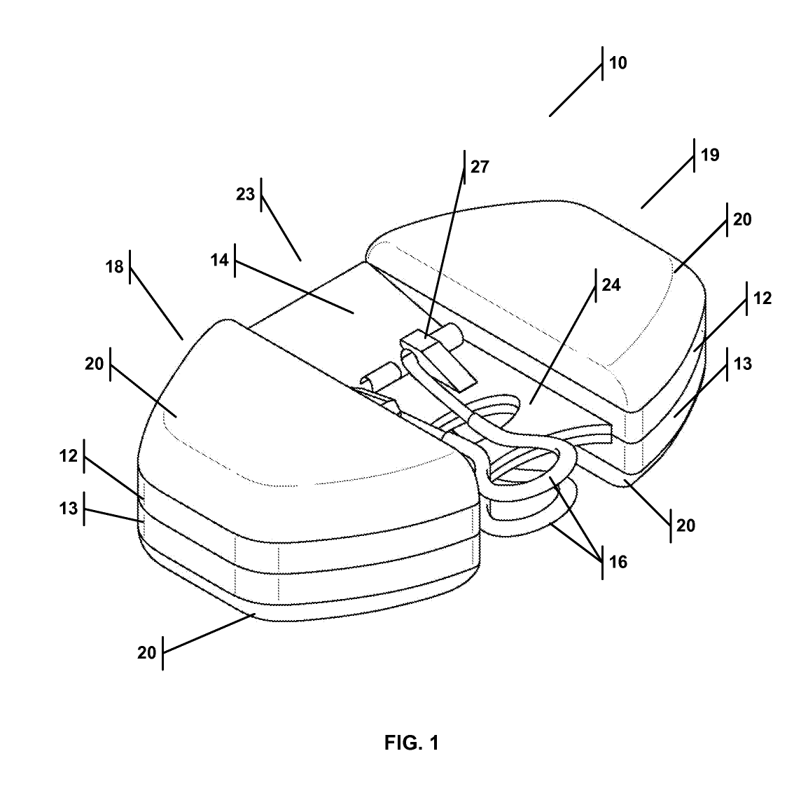

Referring to FIG. 1, the anti-snoring apparatus 10 that is easily and securely attached to a garment 15 or undergarment of the user (FIG. 7). The anti-snoring apparatus 10 makes it uncomfortable for the user to lie on their back and promotes the user to acquire a different position while sleeping and therefore refrain from snoring. The anti-snoring apparatus 10 has a clamshell body with first and second body portions 12, 13. The first and second body portions 12, 13 are secured together by a hinge clip 14. The hinge clip 14 biases or tensions the first and second body portions 12, 13 in a closed position (FIG. 1).

The closed position is where the first and second body portions 12, 13 are held together so they can close on or grip the garment 15 of a user (FIG. 7). The clamshell body is typically secured on the waist band of the user's garment 15. The waist band or clothing does not require any specific structure for the anti-snoring apparatus 10 to be secured thereon. It only requires an edge over which the anti-snoring apparatus 10 may be secured. The edge or waist band is received between the first and second body portions 12, 13 of the apparatus.

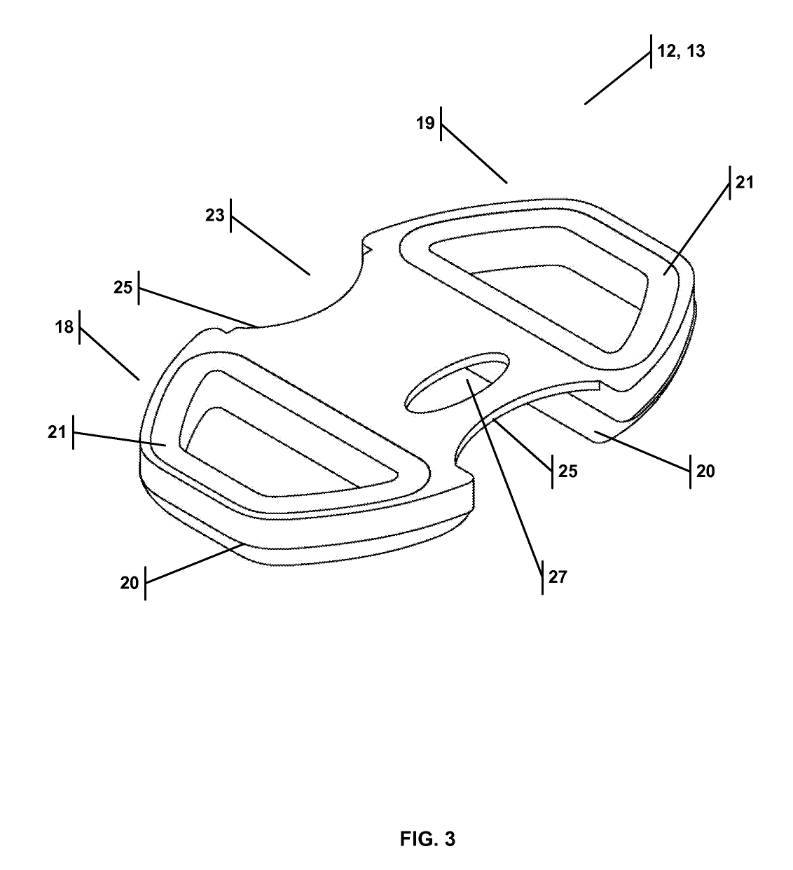

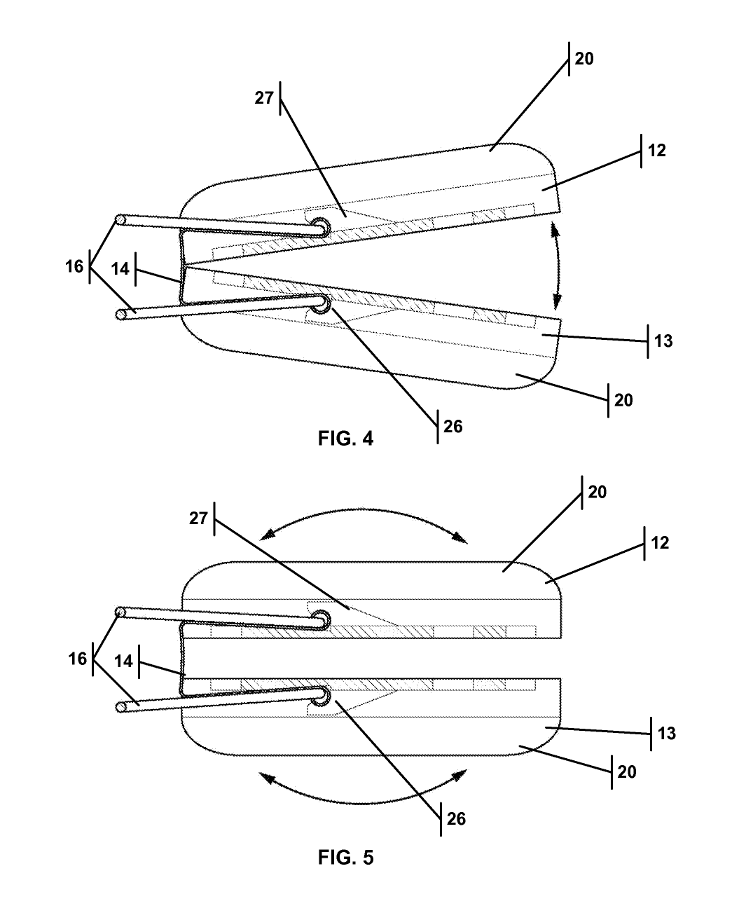

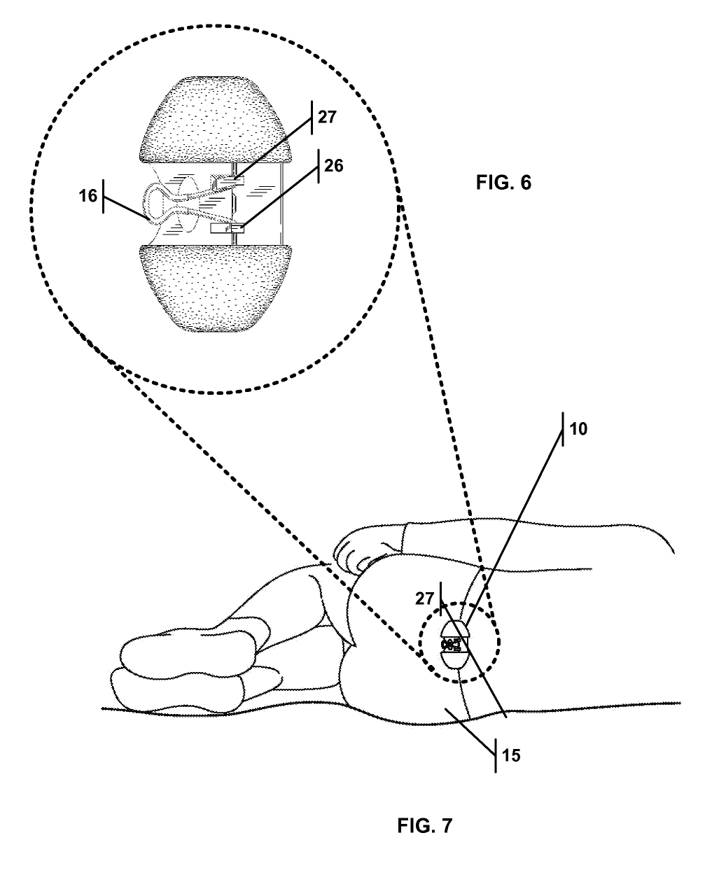

The hinge clip 14 includes two opening members 16, each pivotally secured to a front edge 17 of the hinge clip 14. The opening members 16 are shown in FIG. 1 in a stored position on the anti-snoring apparatus. They can be rotated or moved into this position before or after the clamshell body has been secured to the user's garment. The opening members 16 are shown in an open or operating position in FIG. 4. The user squeezes or urges the opening members 16 together which moves the body portions 12, 13 into an open position as seen in FIG. 4. This enables the user to slide the anti-snoring apparatus 10 over the waistband or edge of the garment 15. Once the user has placed the anti-snoring apparatus 10 on the garment 15, the opening members 16 are released and the hinge clip 14 moves the body portions 12, 13 to the closed position about the garment 15 as seen in FIG. 6.

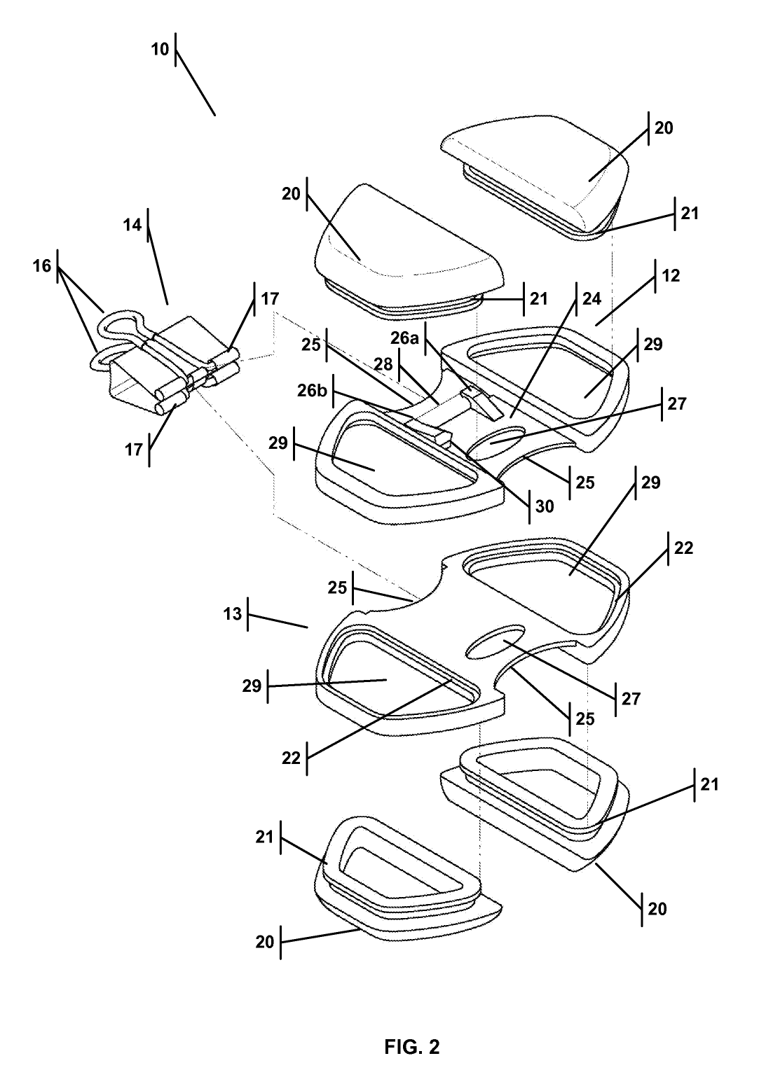

Each of the first and second body portions 12, 13 includes first and second end portions 18, 19. Each of the body portions 12, 13 include at least one pair of extensions 20 that protrude outwardly to increase the height or thickness of the anti-snoring apparatus (FIG. 2). The extensions 20 may be formed with the body portion in one piece such as by co-molding (not shown). The extensions could also be attached to the body portions by adhesive or other means (not shown). The extensions 20 are preferably removably attached to their respective body portion 12, 13. They essentially are inserts that can be removed and replaced by the user. Preferably a plurality of pairs of extensions 20 is provided for each body portion 12, 13 to allow the user to vary the height or thickness of the anti-snoring apparatus 10. The extensions 20 can be made of a material that is of a different hardness than the body portions. The extensions 20 are preferably made of a material that is softer than the body portions 12, 13. The extensions 20 each have a flange 21 or lip that secures them to their respective end 18, 19 of the body portions 12, 13 as seen in FIG. 2.

Each of the first and second end portion include an aperture 29 for receiving part of the extension 20 there through. Each of the first and second end portions 18, 19 is provided with a flange recess 22 to receive the flange 21 as seen in FIGS. 2 and 3 when it has been pressed through its respective aperture 29. The flanges 21 in conjunction with the flange recesses 22 secure the extensions 20 to their respective end portion 18, 19 of body portions 12, 13. The flanges 21, when made of a softer material, provide a gripping surface that helps the apparatus to remain securely in place on the garment 15 of the user. The flanges 21 of the extensions 20 may be coplanar or flush with the inner surface of the body portion or they may extend outwardly away from the inner surface of the body portion (not shown). When outwardly extending, the flanges 21 would be in a position to grip or contact the garment 15 before the body portions 12, 13.

The first and second end portions 18, 19 are joined by a central portion 23 (FIGS. 2 and 3). The central portion 23 may be a web of material that is thinner than the end portions 18, 19. A groove 24 is essentially created above each central portion 23. The grooves 24 allow the hinge clip 14 to be spaced from the outer surfaces of the extensions 20 (FIG. 1). This will prevent the hinge clip 14 from contacting the user when secured to their garment 15. Each central portion 23 or web may be may be provided with pair of notches 25. Each notch 25 is located at front and rear of the central portion 23 making the end portions larger or bulbous with respect to the central portion. This gives the clamshell body and each of the body portions 12, 13 a generally dumbbell shape (FIGS. 1 and 3). It should also be noted that the body portions 12, 13 may be identical to each other to ease in the manufacture of the apparatus.

The notches 25 are shown with a curved surface but this could be a linear surface or straight line. The central portion 23 may include openings 27 therein though they are not essential. Each central portion 23 includes a securing mechanism for receiving the front edges 17 of the hinge clip 14. Each securing mechanism resides in the groove 24 of its respective body portion 12, 13. The securing mechanisms are made as part of or are secured to each web or central portion 23. Each securing mechanism includes first and second protrusions 26a, 26b and may include a channel 28 for receiving a front edge 17 of the hinge clip 14. The first and second protrusions 26a, 26b are spaced apart from each other and may extend over the channel 28 to hold and grip a front edge 17 of the hinge clip 14. The first and second protrusions 26a, 26b may be formed to secure the hinge clip 14 without the need of a channel 28. The first and second protrusions 26a, 26b include rounded or curved portions that receive the rounded front edge 17 of the hinge clip 14.

The securing mechanisms allow the body portions 12, 13 to pivot or rotate with respect the hinge clip 14 as seen in FIGS. 4 and 5. This enables the body portions 12, 13 to remain parallel to each other when they spaced apart as seen in FIG. 5 or when they are secured to a garment (not shown). The parallel spacing of the body portions 12, 13 allows the apparatus to be securely attached to garments of various thicknesses. A locking member 30 may be provided on one of the first and second protrusions 26a, 26b. The locking member 30 secures a respective one of the opening members 16 in the stored position. The locking member 30 is shown on protrusion 26a in FIG. 2 and extends away from protrusion 26a to form a lock or a resilient catch to secure opening member 16 there under. The locking member 30 may be an angled surface as seen in FIG. 6 due to the angled nature of opening members 16. The locking member 30 may act as a resilient catch where the locking member will deform due to the resiliency of the plastic material of which it is made.

The body portions 12, 13, the extensions 20, the attachment mechanisms, and the locking member 30 may be made of a durable plastic or polymer or any other suitable material. The hinge clip 14 may be made of a resilient metal to provide the necessary tension or bias to maintain the body portions 12, 13 in the closed position. It is envisioned that the opening members 16 will be made of durable metal so they do not break easily.

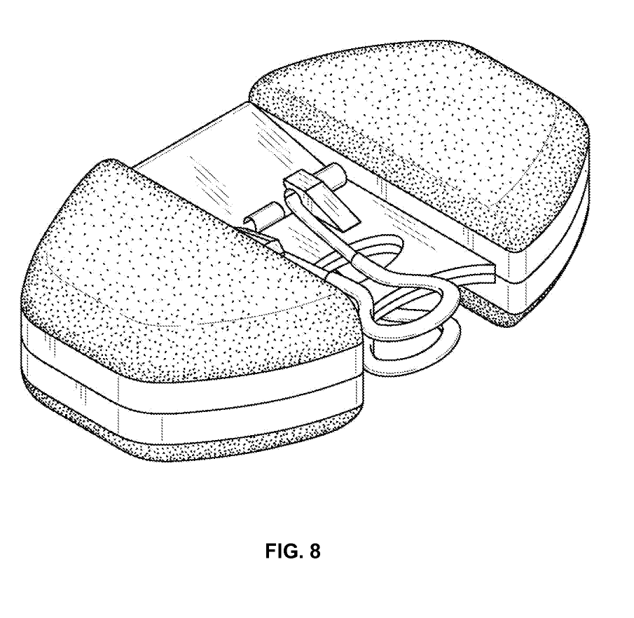

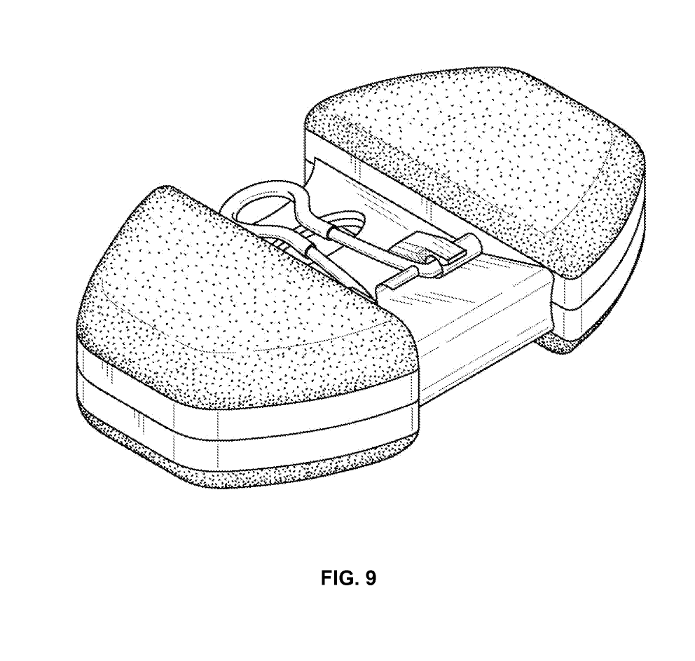

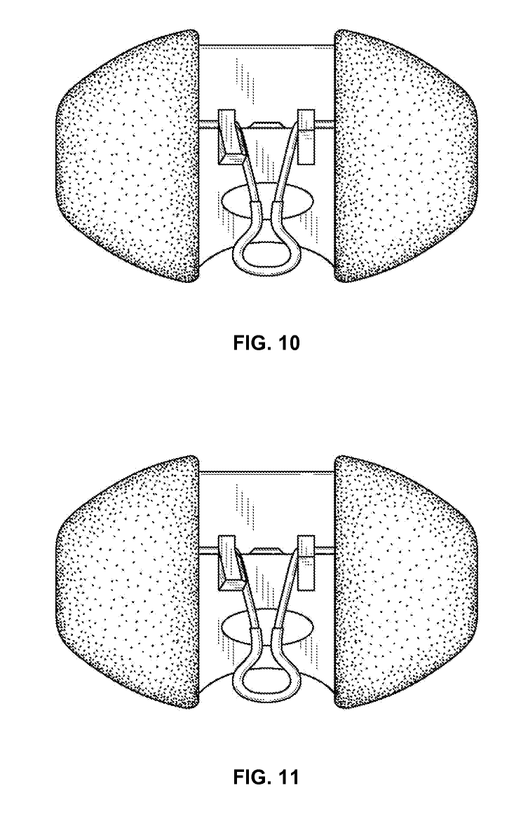

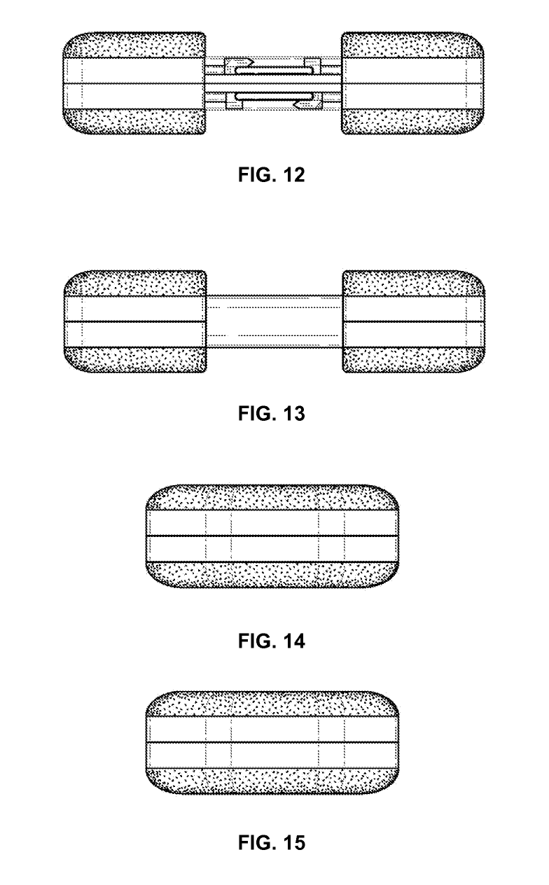

As shown in FIGS. 8-15, an embodiment of the previously described anti-snoring apparatus 10 is shown from various angles.

The invention has been described in connection with specific embodiments that illustrate examples of the invention but do not limit its scope. Various example systems have been shown and described having various aspects and elements. Unless indicated otherwise, any feature, aspect or element of any of these systems may be removed from, added to, combined with or modified by any other feature, aspect or element of any of the systems. As will be apparent to persons skilled in the art, modifications and adaptations to the above-described systems and methods can be made without departing from the spirit and scope of the invention, which is defined only by the following claims. Moreover, the applicant expressly does not intend that the following claims “and the embodiments in the specification to be strictly coextensive.” Phillips v. AHW Corp., 415 F.3d 1303, 1323 (Fed. Cir. 2005) (en banc).

1. An anti-snoring apparatus, the comprising:
a clamshell body with an first body portion and a second body portion;
the first and second body portions secured together in a pivotal relationship by a hinge clip;
the hinge clip biasing the first and second body portions together in a closed position, wherein the clamshell body can be opened and secured on an article of clothing worn by the user to prevent the user from sleeping on their back.

2. The anti-snoring apparatus of claim 1, wherein the hinge clip has first and second opening members pivotally secured thereon allowing the user to move the first and second body portions into an open position.

3. The anti-snoring apparatus of claim 2, wherein each of the first and second body portions includes a groove for receiving the hinge clip and a respective one of said first and second opening members.

4. The anti-snoring apparatus of claim 1, wherein each of the first and second body portions has a central portion with a securing mechanism thereon for retaining the hinge clip.

5. The anti-snoring apparatus of claim 1, wherein each of the first and second body portions has a securing mechanism thereon for retaining an edge of the hinge clip, each said securing mechanism being positioned in a groove of its respective body portion.

6. The anti-snoring apparatus of claim 1, wherein each of the first and second body portions has a pair of extensions that protrude outwardly therefrom.

7. The anti-snoring apparatus of claim 6, wherein each of said pair of extensions is removable from its respective body portion.

8. The anti-snoring apparatus of claim 7, wherein each of said pair of extensions includes a plurality of pairs of extensions, wherein each pair of said plurality of pairs has a different height from the other pairs.

9. The anti-snoring apparatus of claim 1, wherein the hinge clip allows the first and second body portions to remain in a parallel relationship to each other when secured about the article of clothing.

10. The anti-snoring apparatus of claim 1, wherein has the hinge clip has first and second edges, wherein a first edge is pivotally secured to the first body portion and the second edge is pivotally secured to the second body portion.

11. A kit for preventing snoring, the kit comprising:
a clamshell body with an first body portion and a second body portion;
the first and second body portions secured together in a pivotal relationship by a hinge clip;
the hinge clip biasing the first and second body portions together in a closed position;
each of the first and second body portions having a pair of extensions that protrude outwardly therefrom, each of said pair of extensions is removable from its respective body portion, each of said pair of extensions includes a plurality of pairs of extensions;
each pair of said plurality of pairs has a different height from the other pairs; and wherein the clamshell can be opened and secured on an article of clothing worn by the user.

12. The kit of claim 11, wherein the hinge clip has first and second opening members pivotally secured thereon allowing the user to move the first and second body portions into an open position.

13. The kit of claim 11, wherein each of the first and second body portions includes a groove for receiving the hinge clip and a respective one of said first and second opening members.

14. The kit of claim 11, wherein each of the first and second body portions has a central portion with a securing mechanism thereon for retaining the hinge clip.

15. The kit of claim 11, wherein each of the first and second body portions includes a securing mechanism thereon for retaining an edge of the hinge clip, each said securing mechanism being positioned in a groove of its respective body portion.

16. The kit of claim 11, wherein the hinge clip allows the first and second body portions to remain in a parallel relationship to each other when secured about the article of clothing.

17. The kit of claim 11, wherein has the hinge clip has first and second edges, wherein a first edge is pivotally secured to the first body portion and the second edge is pivotally secured to the second body portion.

18. A method of preventing snoring, the method comprising:
providing a clamshell body with an first body portion and a second body portion;
securing the first and second body portions together in a pivotal relationship with a hinge clip, the hinge clip biasing the body portions together in a closed position;
providing an article of clothing with an edge or waist band;
opening the clamshell body and securing the body on the edge or waist band; and
wherein the article of clothing is worn by the user with the body there attached to prevent snoring.

19. The method of claim 18, providing each of the first and second body portions with a pair of extensions that protrude outwardly therefrom.

20. The method of claim 19, providing each of said pair of extensions includes a plurality of pairs of extensions, wherein each pair of said plurality of pairs has a different height from the other pairs.

21. The method of claim 18, providing the hinge clip allows the first and second body portions to remain in a parallel relationship to each other when secured about the article of clothing.
